(19) United States
(12) Patent Application Publication
(10) Pub. No.: US 2014/0012151 A1
(43) Pub. Date: Jan. 9, 2014

(54) Method And System For Displaying EEG Data

(71) Applicants: Nicolas Nierenberg, La Jolla, CA (US); Scott B. Wilson, Del Mar, CA (US); Mark Scheuer, Wexford, PA (US)

(72) Inventors: Nicolas Nierenberg, La Jolla, CA (US); Scott B. Wilson, Del Mar, CA (US); Mark Scheuer, Wexford, PA (US)

(73) Assignee: PERSYST DEVELOPMENT CORPORATION, San Diego, CA (US)

(21) Appl. No.: 13/542,665

(22) Filed: Jul. 6, 2012

(51) Int. Cl. A61B 5/0476

(52) U.S. Cl. 600/544

(57) ABSTRACT

A method and system for displaying EEG data is disclosed herein. A processed EEG report is overlayed on an original EEG report to generate a combined EEG report, wherein an x-axis of the processed EEG report is aligned with an x-axis of the original EEG report, and wherein a y-axis of the processed EEG report is aligned with an y-axis of the original EEG report. The combined EEG report is displayed wherein the processed EEG report is visually distinctive from the original EEG report.

### CROSS REFERENCES TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of the Invention

The present invention generally relates to a method and system for displaying EEG data. More specifically, the present invention relates to a method and system for displaying a combined EEG reporting comprising a processed EEG report overlayed on an original EEG report.

2. Description of the Related Art

An electroencephalogram (“EEG”) is a diagnostic tool that measures and records the electrical activity of a person's brain in order to evaluate cerebral functions. Multiple electrodes are attached to a person's head and connected to a machine by wires. The machine amplifies the signals and records the electrical activity of a person's brain. The electrical activity is produced by the summation of neural activity across a plurality of neurons. These neurons generate small electric voltage fields. The aggregate of these electric voltage fields create an electrical reading which electrodes on the person's head are able to detect and record. An EEG is a superposition of multiple simpler signals. In a normal adult, the amplitude of an EEG signal typically ranges from 1 micro-Volt to 100 micro-Volts, and the EEG signal is approximately 10 to 20 milli-Volts when measured with subdural electrodes. The monitoring of the amplitude and temporal dynamics of the electrical signals provides information about the underlying neural activity and medical conditions of the person.

An EEG is performed to: diagnose epilepsy; verify problems with loss of consciousness or dementia; verify brain activity for a person in a coma; study sleep disorders, monitor brain activity during surgery, and additional physical problems.

Multiple electrodes (typically 17-21, however there are standard positions for at least 70) are attached to a person's head during an EEG. The electrodes are referenced by the position of the electrode in relation to a lobe or area of a person's brain. The references are as follows: F=frontal; Fp=frontopolar; T=temporal; C=central; P=parietal; O=occipital; and A=auricular (ear electrode). Numerals are used to further narrow the position and “z” points relate to electrode sites in the midline of a person's head. An electrocardiogram (“EKG”) may also appear on an EEG display.

The EEG records brain waves from different amplifiers using various combinations of electrodes called montages. Montages are generally created to provide a clear picture of the spatial distribution of the EEG across the cortex. A montage is an electrical map obtained from a spatial array of recording electrodes and preferably refers to a particular combination of electrodes examined at a particular point in time.

In bipolar montages, consecutive pairs of electrodes are linked by connecting the electrode input 2 of one channel to input 1 of the subsequent channel, so that adjacent channels have one electrode in common. The bipolar chains of electrodes may be connected going from front to back (longitudinal) or from left to right (transverse). In a bipolar montage signals between two active electrode sites are compared resulting in the difference in activity recorded. Another type of montage is the referential montage or monopolar montage. In a referential montage, various electrodes are connected to input 1 of each amplifier and a reference electrode is connected to input 2 of each amplifier. In a reference montage, signals are collected at an active electrode site and compared to a common reference electrode.

Reference montages are good for determining the true amplitude and morphology of a waveform. For temporal electrodes, CZ is usually a good scalp reference.

Being able to locate the origin of electrical activity (“localization”) is critical to being able to analyze the EEG. Localization of normal or abnormal brain waves in bipolar montages is usually accomplished by identifying “phase reversal,” a deflection of the two channels within a chain pointing to opposite directions. In a referential montage, all channels may show deflections in the same direction. If the electrical activity at the active electrodes is positive when compared to the activity at the reference electrode, the deflection will be downward. Electrodes where the electrical activity is the same as at the reference electrode will not show any deflection. In general, the electrode with the largest upward deflection represents the maximum negative activity in a referential montage.

Some patterns indicate a tendency toward seizures in a person. A physician may refer to these waves as “epileptiform abnormalities” or “epilepsy waves.” These include spikes, sharp waves, and spike-and-wave discharges. Spikes and sharp waves in a specific area of the brain, such as the left temporal lobe, indicate that partial seizures might possibly come from that area. Primary generalized epilepsy, on the other hand, is suggested by spike-and-wave discharges that are widely spread over both hemispheres of the brain, especially if they begin in both hemispheres at the same time.

There are several types of brain waves: alpha waves, beta waves, delta wave, theta waves and gamma waves. Alpha waves have a frequency of 8 to 12 Hertz (“Hz”). Alpha waves are normally found when a person is relaxed or in a waking state when a person's eyes are closed but the person is mentally alert. Alpha waves cease when a person's eyes are open or the person is concentrating. Beta waves have a frequency of 13 Hz to 30 Hz. Beta waves are normally found when a person is alert, thinking, agitated, or has taken high doses of certain medicines. Delta waves have a frequency of less than 3 Hz. Delta waves are normally found only when a person is asleep (non-REM or dreamless sleep) or the person is a young child. Theta waves have a frequency of 4 Hz to 7 Hz. Theta waves are normally found only when the person is asleep (dream or REM sleep) or the person is a young child. Gamma waves have a frequency of 30 Hz to 100 Hz. Gamma waves are normally found during higher mental activity and motor functions.

The following definitions are used herein.

“Amplitude” refers to the vertical distance measured from the trough to the maximal peak (negative or positive). It expresses information about the size of the neuron population and its activation synchrony during the component generation.

The term “analogue to digital conversion” refers to when an analogue signal is converted into a digital signal which can then be stored in a computer for further processing. Analogue signals are “real world” signals (e.g., physiological signals such as electroencephalogram, electrocardiogram or electrooculogram). In order for them to be stored and manipulated by a computer, these signals must be converted into a discrete digital form the computer can understand.

“Artifacts” are electrical signals detected along the scalp by an EEG, but that originate from non-cerebral origin. There are patient related artifacts (e.g., movement, sweating, ECG, eye movements) and technical artifacts (50/60 Hz artifact, cable movements, electrode paste-related).

The term “differential amplifier” refers to the key to electrophysiological equipment. It magnifies the difference between two inputs (one amplifier per pair of electrodes).

“Duration” is the time interval from the beginning of the voltage change to its return to the baseline. It is also a measurement of the synchronous activation of neurons involved in the component generation.

“Electrode” refers to a conductor used to establish electrical contact with a nonmetallic part of a circuit. EEG electrodes are small metal discs usually made of stainless steel, tin, gold or silver covered with a silver chloride coating. They are placed on the scalp in special positions.

“Electrode gel” acts as a malleable extension of the electrode, so that the movement of the electrodes leads is less likely to produce artifacts. The gel maximizes skin contact and allows for a low-resistance recording through the skin.

The term “electrode positioning” (10/20 system) refers to the standardized placement of scalp electrodes for a classical EEG recording. The essence of this system is the distance in percentages of the 10/20 range between Nasion-Inion and fixed points. These points are marked as the Frontal pole (Fp), Central (C), Parietal (P), occipital (O), and Temporal (T). The midline electrodes are marked with a subscript z, which stands for zero. The odd numbers are used as subscript for points over the left hemisphere, and even numbers over the right

“Electroencephalogram” or “EEG” refers to the tracing of brain waves, by recording the electrical activity of the brain from the scalp, made by an electroencephalograph.

“Electroencephalograph” refers to an apparatus for detecting and recording brain waves (also called encephalograph).

“Epileptiform” refers to resembling that of epilepsy.

“Filtering” refers to a process that removes unwanted frequencies from a signal.

“Filters” are devices that alter the frequency composition of the signal.

“Montage” means the placement of the electrodes. The EEG can be monitored with either a bipolar montage or a referential one. Bipolar means that there are two electrodes per one channel, so there is a reference electrode for each channel. The referential montage means that there is a common reference electrode for all the channels.

“Morphology” refers to the shape of the waveform. The shape of a wave or an EEG pattern is determined by the frequencies that combine to make up the waveform and by their phase and voltage relationships. Wave patterns can be described as being: “Monomorphic”. Distinct EEG activity appearing to be composed of one dominant activity. “Polymorphic”. distinct EEG activity composed of multiple frequencies that combine to form a complex waveform. “Sinusoidal”. Waves resembling sine waves. Monomorphic activity usually is sinusoidal. “Transient”. An isolated wave or pattern that is distinctly different from background activity.

“Spike” refers to a transient with a pointed peak and a duration from 20 to under 70 msec.

The term “sharp wave” refers to a transient with a pointed peak and duration of 70-200 msec.

The term “neural network algorithms” refers to algorithms that identify sharp transients that have a high probability of being epileptiform abnormalities.

“Noise” refers to any unwanted signal that modifies the desired signal. It can have multiple sources.

“Periodicity” refers to the distribution of patterns or elements in time (e.g., the appearance of a particular EEG activity at more or less regular intervals). The activity may be generalized, focal or lateralized.

An EEG epoch is an amplitude of a EEG signal as a function of time and frequency.

Various techniques have been developed to present the EEG data to a physician or technician. However, these techniques are still lacking If the raw EEG report is presented to a physician or technician, then artifacts typically render the EEG report incapable of distinguishing brain activity such as a seizure from artifacts. Despite the use of artifact reduction algorithms, the failure to accurately distinguish true physiological rhythmicity from the artifacts is a serious shortcoming of current software systems and requires an expert assessment. What is needed is a way for a physician or technician to clearly compare the raw EEG report with a filtered report in order to better analyze the patient's brain activity. Visually inspecting a raw EEG and then reviewing a processed EEG is difficult since the y-axis and X-axis can be misplaced due to various processing techniques such as stitching. Further, channels may be overlapped adding to further difficulty in reading an EEG.

### BRIEF SUMMARY OF THE INVENTION

The present invention provides an EEG system and method that overlays a processed EEG report over a raw EEG report to permit a physician or technician to clearly see the activity reported.

One aspect of the present invention is a method for displaying EEG data. The method includes generating an original EEG report from an EEG signal. The original EEG report is generated from an EEG machine comprising a plurality of electrodes and processor. The original EEG report comprises a first plurality of channels. The method also includes performing artifact reduction on the original EEG signal to generate a processed EEG report. The processed EEG report comprises a second plurality of channels. The method also includes overlaying the processed EEG report on the original EEG report to generate a combined EEG report. An x-axis of the processed EEG report is aligned with an x-axis of the original EEG report. A y-axis of the processed EEG report is aligned with an y-axis of the original EEG report. The first plurality of channels of the original EEG report are equal to the second plurality of channels of the processed EEG report. The method also includes displaying the combined EEG report wherein the processed EEG report is visually distinctive from the original EEG report. An activity at a specific time on one channel of the first plurality of channels of the original EEG report is identifiable on a corresponding channel of the second plurality of channels of the processed EEG report at the specific time. The activity is preferably spikes, sharp waves, spike and wave discharges, artifacts, and the like.

Another aspect of the present invention is a method for displaying a combined EEG report. The method includes generating an original EEG report from an EEG signal. The original EEG report is generated from an EEG machine comprising a plurality of electrodes and processor. The original EEG report comprises a first plurality of channels. The method also includes performing artifact reduction on the original EEG signal to generate a processed continuous EEG report. The processed EEG report comprises a second plurality of channels. The method also includes overlaying the processed continuous EEG report on the original EEG report to generate a combined EEG report. An x-axis of the processed continuous EEG report is aligned with an x-axis of the original EEG report. A y-axis of the processed continuous EEG report is aligned with an y-axis of the original EEG report. The first plurality of channels of the original EEG report are equal to the second plurality of channels of the processed continuous EEG report. The method also includes displaying the combined EEG report wherein the processed EEG report is visually distinctive from the original EEG report. An activity at a specific time on one channel of the first plurality of channels of the original EEG report is identifiable on a corresponding channel of the second plurality of channels of the processed continuous EEG report at the specific time.

Yet another aspect of the present invention is a system for displaying EEG data. The system includes a patient component, a machine component and a display screen. The patient component comprises a plurality of electrodes for generating an EEG signal. The EEG machine component comprises an amplifier and a processor. The processor is configured to generate an original EEG report from an EEG signal. The original EEG report comprises a first plurality of channels. The processor is also configured to perform artifact reduction on the original EEG signal to generate a processed EEG report. The processed EEG report comprises a second plurality of channels. The processor is also configured to overlay the processed EEG report on the original EEG report to generate a combined EEG report. An x-axis of the processed EEG report is aligned with an x-axis of the original EEG report. A y-axis of the processed EEG report is aligned with an y-axis of the original EEG report. The first plurality of channels of the original EEG report are equal to the second plurality of channels of the processed EEG report. The display screen displays the combined EGG report wherein the processed EEG report is visually distinctive from the original EEG report, and wherein an activity at a specific time on one channel of the first plurality of channels of the original EEG report is identifiable on a corresponding channel of the second plurality of channels of the processed EEG report at the specific time.

Having briefly described the present invention, the above and further objects, features and advantages thereof will be recognized by those skilled in the pertinent art from the following detailed description of the invention when taken in conjunction with the accompanying drawings.

### BRIEF DESCRIPTION OF THE SEVERAL VIEWS OF THE DRAWINGS

FIG. 1 is an illustration of a portion of a raw EEG report having nineteen channels.

FIG. 1A is an enlargement of circle 1A of FIG. 1.

FIG. 2 is an illustration of a portion of a processed EEG report having nineteen channels.

FIG. 2A is an enlargement of circle 2A of FIG. 2.

FIG. 3 is an illustration of a portion of a processed continuous EEG report in which sections of the epochs of the EEG report are stitched to overlap.

FIG. 3A is an enlargement of circle 3A of FIG. 3.

FIG. 4 is an illustration of a portion of a combined EEG report having a processed EEG report overlay on a raw EEG report.

FIG. 4A is an enlargement of circle A of FIG. 4.

FIG. 4B is an enlargement of circle B of FIG. 4.

FIG. 4C is an enlargement of circle C of FIG. 4.

FIG. 5 is a flow chart of a method for displaying EEG data.

FIG. 6 is a flow chart for a method of artifact reduction.

FIG. 7 is an illustration of an EEG system used on a patient.

FIG. 8 is a map representing the international 10-20 electrode system for electrode placement for an EEG.

FIG. 9 is a detailed map representing the intermediate 10% electrode positions, as standardized by the American Electroencephalographic Society, for electrode placement for an EEG.

FIG. 10 is a block diagram of an EEG machine component of a EEG system.

### DETAILED DESCRIPTION OF THE INVENTION

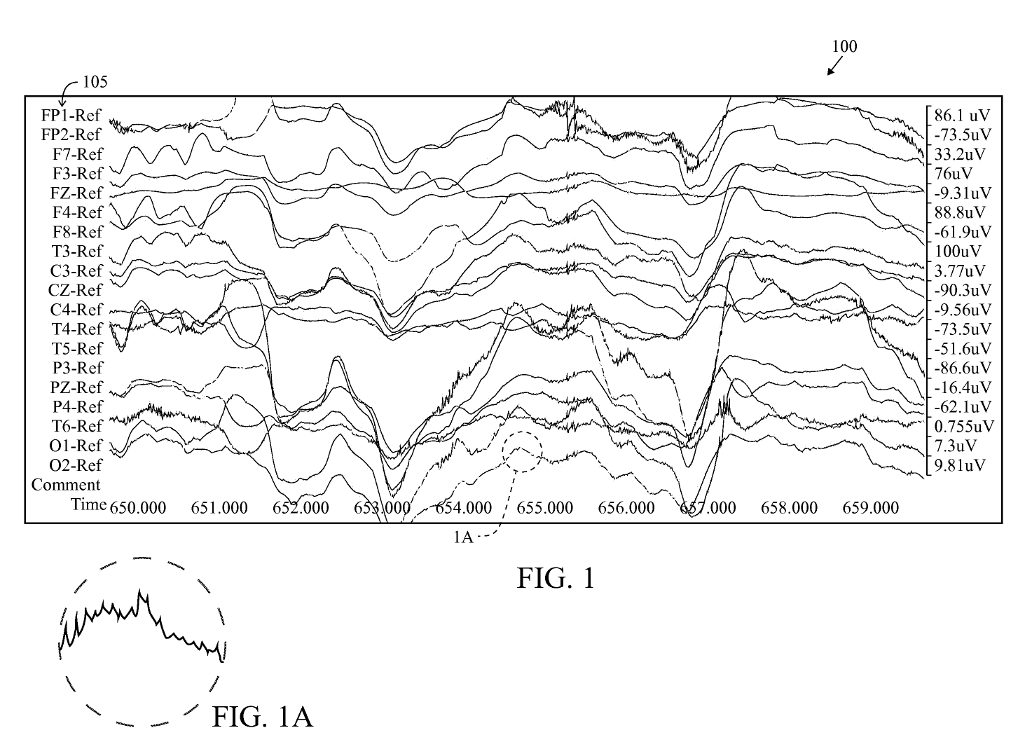

A raw or original EEG report 100 is shown in FIG. 1. The original EEG report 100 has a plurality of channels FP1-Ref through to O2-Ref, shown at the Y axis 105 of the report. The X-axis of the report is time. The original EEG report 100 has not been subjected to artifact reduction. The original EEG report contains artifacts from various sources such as muscle movement, eye movement, sweating, electrode cables and the like. However, the EEG may also have certain activity that a physician or technician is looking for from the EEG report in order to accurately analyze the patient's brain activity. For example, the activity shown in FIG. 1A at a time 655.000 may represent a certain stage of brain activity for the patient that is important to the physician or technician. However, normally, the physician or technician will not review the raw EEG report 100 due to the presence of artifacts.

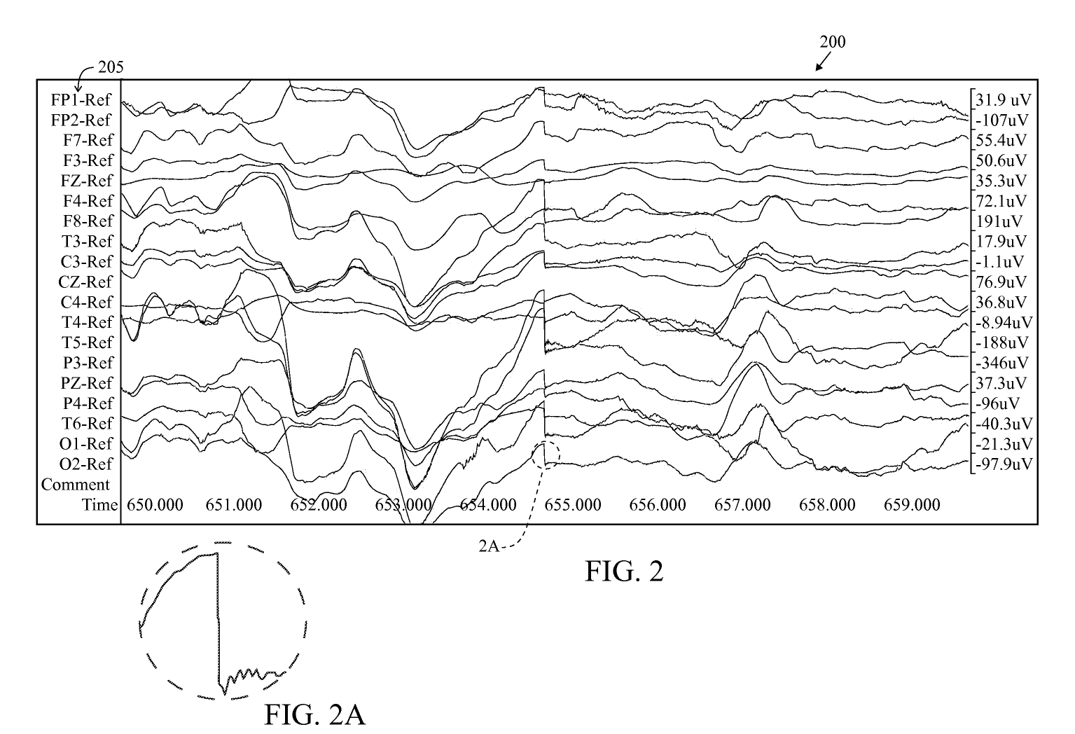

FIG. 2 is an illustration of a processed EEG report 200 of the original EEG report 100 of FIG. 1 that has undergone artifact reduction and the stitching of epochs in order to recreate the EEG report. The processed EEG report 200 has a plurality of channels FP1-Ref through to O2-Ref, shown at the Y axis 205 of the report. The X-axis of the report is time. As shown in FIG. 2A, the processed EEG report 200 at time 655.000 is quite different in appearance than the original EEG report 100 at time 655.000. This primarily due to stitching of epochs to recreate the EEG report, however if a physician or technician was only looking at the processed EEG report 200, the physician or technician would not be aware of the true activity at time 655.000.

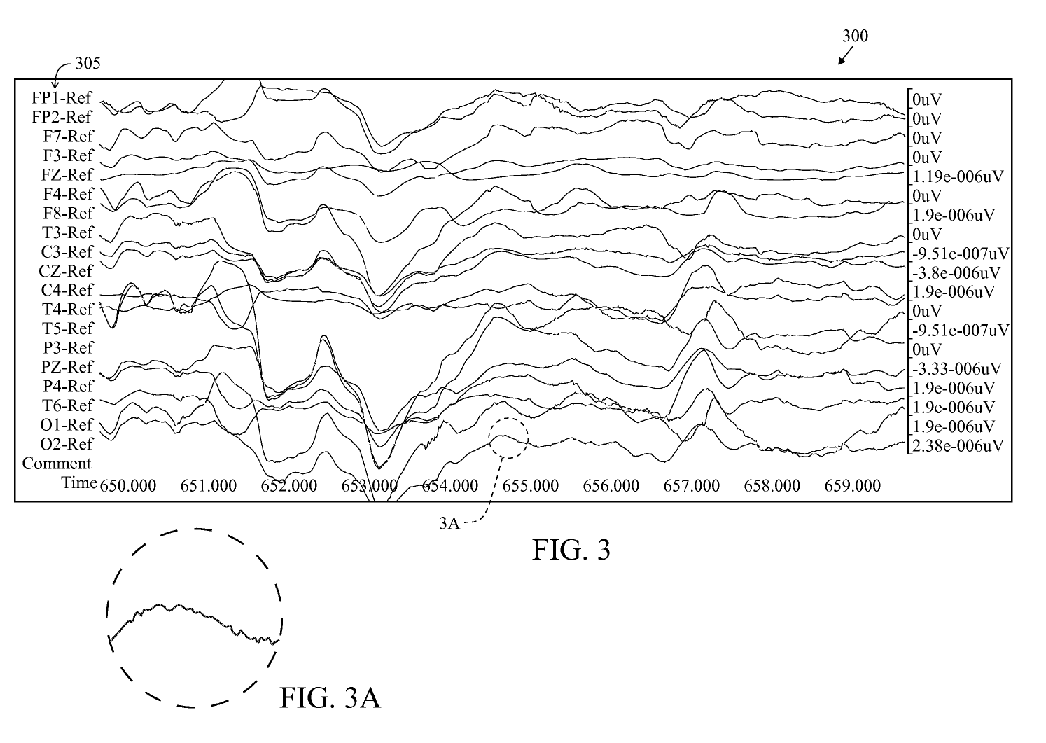

FIG. 3 is an illustration of a processed continuous EEG report 300 of the original EEG report 100 of FIG. 1 that has undergone artifact reduction and the stitching of overlapping epochs in order to recreate the EEG report. The processed EEG report 300 has a plurality of channels FP1-Ref through to O2-Ref, shown at the Y axis 305 of the report. The X-axis of the report is time. As shown in FIG. 3A, the processed EEG report 300 at time 655.000 is more similar in appearance to the original EEG report 100 at time 655.000 than the processed EEG report 200 of FIG. 2. However, there is still difficulty in analyzing a patient's brain activity by switching back and forth from an original EEG report 100 to a processed EEG report 200 or a processed continuous EEG report 300.

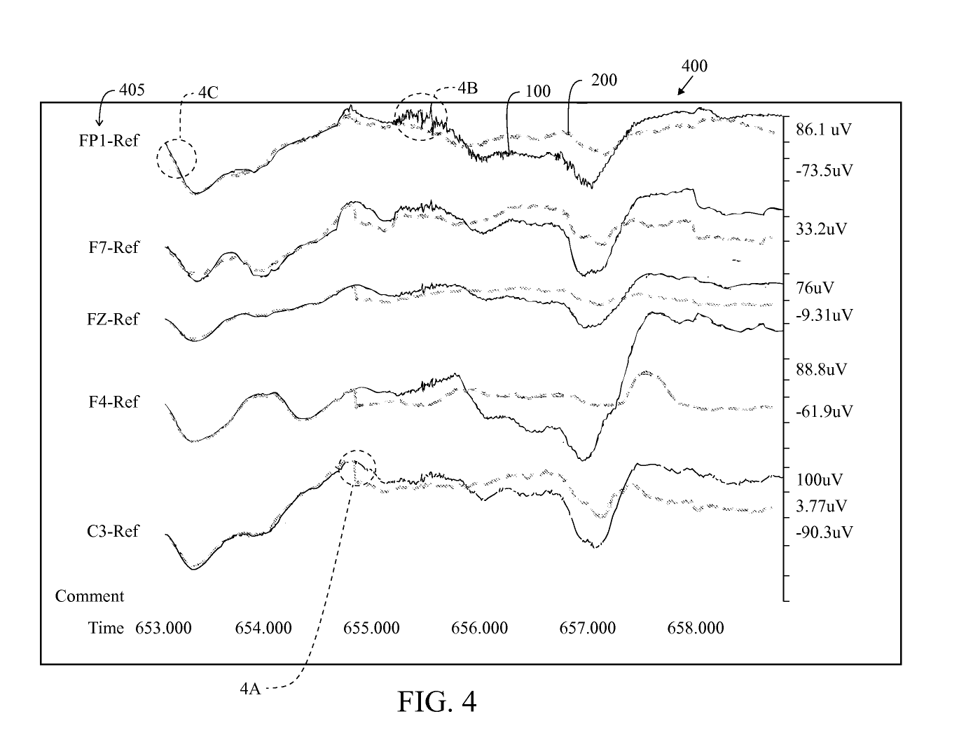

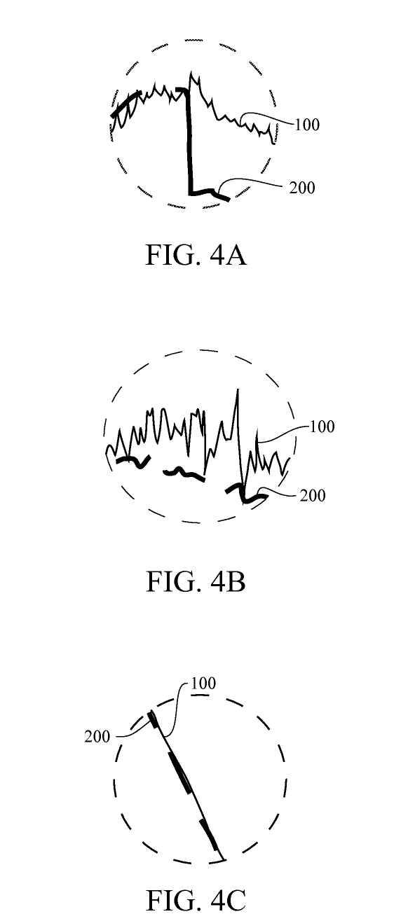

FIG. 4 is an illustration of a combined EEG report 400 comprising the original EEG report 100 and the processed EEG report 200. The illustration of the combined EEG report 400 only has five channels in order to clearly illustrate the invention, however, those skilled in the pertinent will recognize that the combined EEG report 400 could have sixteen, twenty, twenty-seven and any number of channels without departing from the scope and spirit of the present invention.

As shown in FIGS. 4, 4A, 4B and 4C, the original EEG report 100 has a first line style and the processed EEG report 200 has a second line style distinctive from the first line style in order to allow a physician and technician to easily and visually distinguish between the original EEG report 100 and the processed EEG report 200. In an alternative embodiment, the original EEG report 100 has a first color (e.g., blue) and the processed EEG report 200 has a second color (e.g. red) distinctive from the first color in order to allow a physician and technician to easily and visually distinguish between the original EEG report 100 and the processed EEG report 200.

As shown in FIG. 4 and specifically FIG. 4C, the channels of the original EEG report 100 are aligned with the channels of the processed EEG report 200 in order to have y-axis alignment.

As shown in FIG. 4 and specifically in FIG. 4A, the x-axis of the original EEG report 100 are aligned with the x-axis of the processed EEG report 200 in order to have time alignment of the two EEG reports in the combined EEG report 400.

Further, the amplitudes for both the original EEG report 100 and the processed EEG report 200 are contained within each of the channels in order to prevent overlapping of the signals.

As shown in FIG. 4B, the original EEG report 100 is quite different from the processed EEG report 200 and a physician or technician may be interested in the activity shown in the original EEG report 100 as compared to the processed EEG report 200.

Those skilled in the pertinent art will recognize that processed continuous EEG report 300 may be substituted for processed EEG report in FIG. 4 in order to demonstrate a comparison between the original EEG report 100 and the processed continuous EEG report 300.

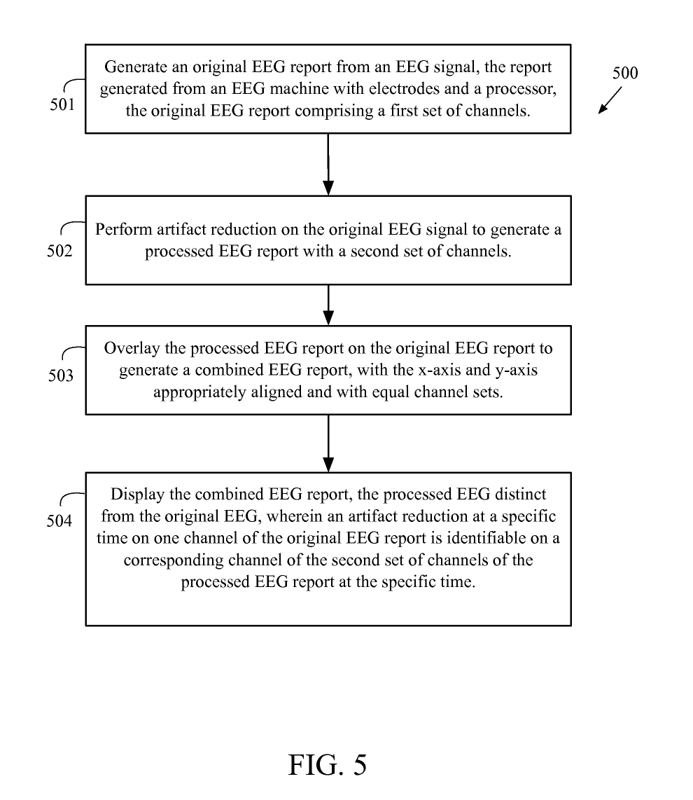

A flow chart for a method 500 for displaying EEG data is shown in FIG. 5. At block 501, an original EEG report is generated from an EEG signal. The original EEG report is generated from an EEG machine comprising a plurality of electrodes and processor. The original EEG report comprises a first plurality of channels. At block 502, artifact reduction is performed on the original EEG signal to generate a processed EEG report. The processed EEG report comprises a second plurality of channels. At block 503, the processed EEG report overlays the original EEG report to generate a combined EEG report. An x-axis of the processed EEG report is aligned with an x-axis of the original EEG report. A y-axis of the processed EEG report is aligned with an y-axis of the original EEG report. The first plurality of channels of the original EEG report are equal to the second plurality of channels of the processed EEG report. At block 504, the combined EEG report is displayed on a display screen, preferably a monitor. The processed EEG report is visually distinctive from the original EEG report. An activity at a specific time on one channel of the first plurality of channels of the original EEG report is identifiable on a corresponding channel of the second plurality of channels of the processed EEG report at the specific time. The activity is preferably spikes, sharp waves, spike and wave discharges, artifacts, and the like

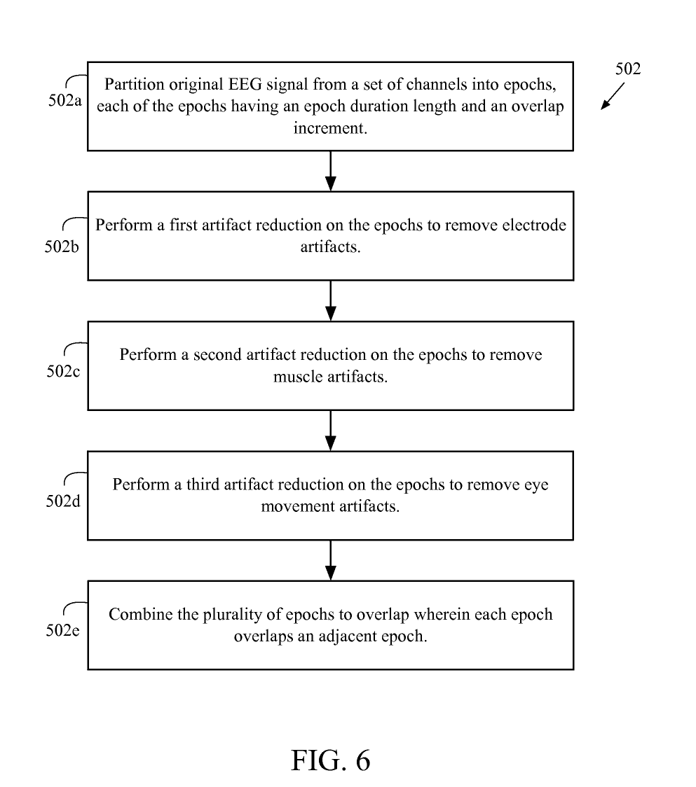

FIG. 6 is a flow chart of a preferred method 502 for artifact reduction of raw EEG data. At block 502a, the original EEG signal is portioned from a set of channels into a plurality of epochs. Each of the plurality of epochs having an epoch duration length and an overlap increment. At block 502b, a first artifact reduction is performed on the plurality of epochs to remove electrode artifacts. At block 502c, a second artifact reduction is performed on the plurality of epochs to remove muscle artifacts. At block 502d, a third artifact reduction is performed on the plurality of epochs to remove eye movement artifacts. At block 502e, the plurality of epochs are combined to overlap wherein each epoch of the plurality of epochs overlaps an adjacent epoch to form a processed continuous EEG report.

Each of the plurality of epochs has an epoch duration length of two seconds and an increment of one second. Alternatively, each of the plurality of epochs has an epoch duration length of four seconds and an increment of two seconds. The artifact removal algorithm is preferably a blind source separation algorithm. The blind source separation algorithm is preferably a CCA algorithm or an ICA algorithm. The clean epochs are preferably combined using a weighted average and the weight of the weighted average is preferably proportional to the ratio of the distance to an epoch center.

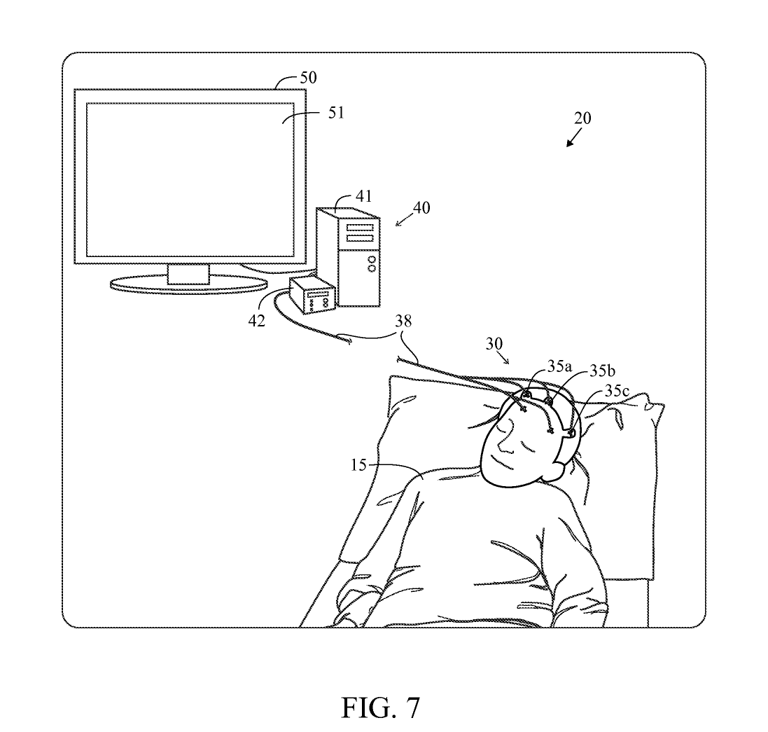

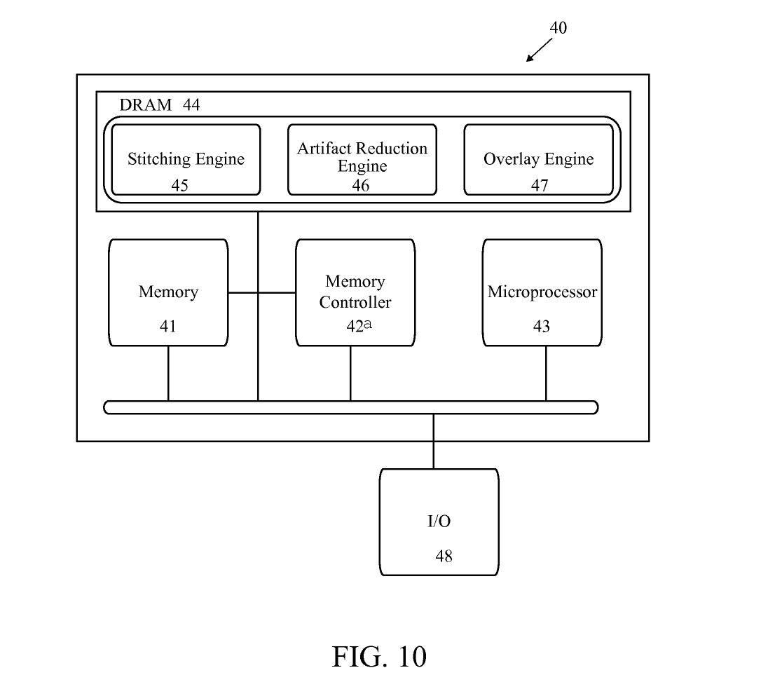

As shown in FIG. 7, an EEG system is generally designated 20. The system preferably includes a patient component 30, an EEG machine component 40 and a display component 50. The patient component 30 includes a plurality of electrodes 35a, 35b, 35c attached to the patient 15 and wired by cables 38 to the EEG machine component 40. The EEG machine component 40 comprises a CPU 41 and an amplifier component 42. The EEG machine component 40 is connected to the display component 50 for display of the combined EEG reports, and for switching from a processed EEG report to the combined EEG reports, or from the processed EEG report to an original EEG report. As shown in FIG. 10, the EEG machine component 40 preferably includes a stitching engine 45, an artifact reduction engine 46, an overlay engine 47, a memory 41, a memory controller 42, a microprocessor 43, a DRAM 44, and an Input/Output 48. Those skilled in the pertinent art will recognize that the machine component 40 may include other components without departing from the scope and spirit of the present invention.

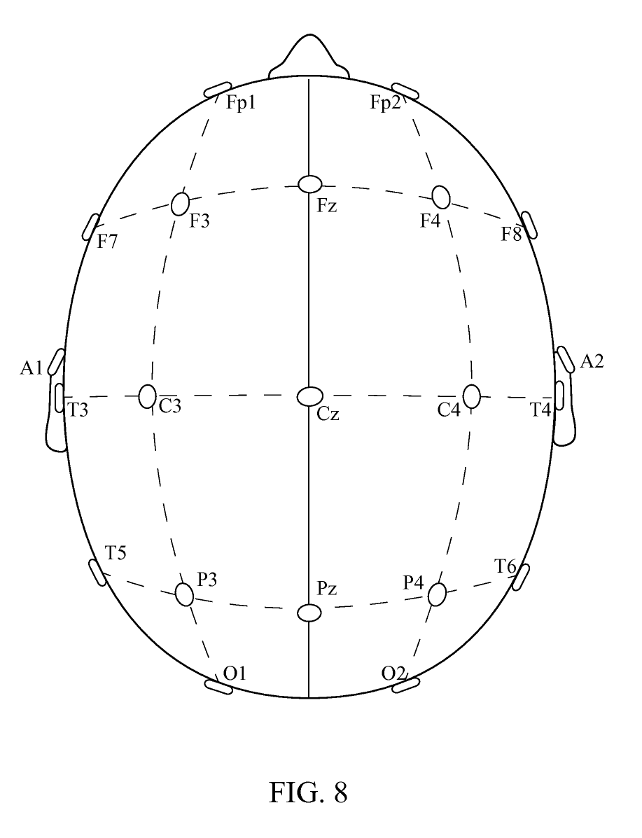

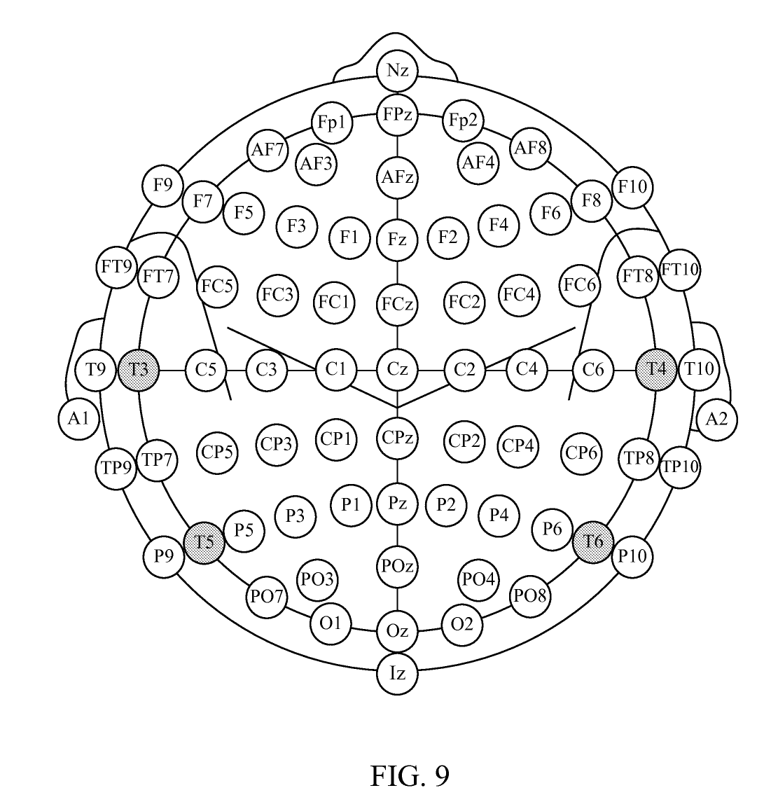

A patient has a plurality of electrodes attached to the patient's head with wires from the electrodes connected to an amplifier for amplifying the signal to a processor which is used to analyze the signals from the electrodes and create an EEG recording. The brain produces different signals at different points on a patient's head. Multiple electrodes are positioned on a patient's head as shown in FIGS. 8 and 9. For example Fp1 on FIG. 8 is represented in channel FP1-Ref on FIG. 4. The number of electrodes determines the number of channels for an EEG. A greater number of channels produces a more detailed representation of a patient's brain activity. Preferably, each amplifier of an EEG machine component 40 corresponds to two electrodes attached to a patient's head. The output from an EEG machine component is the difference in electrical activity detected by the two electrodes. The placement of each electrode is critical for an EEG report since the closer electrode pairs are to each other, the less difference in the brainwaves that are recorded by the EEG machine component. A more thorough description of an electrode utilized with the present invention is detailed in Wilson et al., U.S. Pat. No. 8,112,141 for a Method And Device For Quick Press On EEG Electrode, which is hereby incorporated by reference in its entirety. The EEG is optimized for automated artifact filtering. The EEG recordings are then processed using neural network algorithms to generate a processed EEG recording which is analyzed for display.

Algorithms for removing artifact from EEG typically use Blind Source Separation (BSS) algorithms like CCA (canonical correlation analysis) and ICA (Independent Component Analysis) to transform the signals from a set of channels into a set of component waves or “sources.” The sources that are judged as containing artifact are removed and the rest of the sources are reassembled into the channel set.

From the foregoing it is believed that those skilled in the pertinent art will recognize the meritorious advancement of this invention and will readily understand that while the present invention has been described in association with a preferred embodiment thereof, and other embodiments illustrated in the accompanying drawings, numerous changes modification and substitutions of equivalents may be made therein without departing from the spirit and scope of this invention which is intended to be unlimited by the foregoing except as may appear in the following appended claim. Therefore, the embodiments of the invention in which an exclusive property or privilege is claimed are defined in the following appended claims.

1. A method for displaying EEG data, the method comprising:
generating an original EEG report from an EEG signal, the original EEG report generated from an EEG machine comprising a plurality of electrodes and processor, the original EEG report comprising a first plurality of channels;
performing artifact reduction on the original EEG signal to generate a processed EEG report, the processed EEG report comprising a second plurality of channels, wherein the processed EEG report is without discontinuities, wherein performing artifact reduction comprises partitioning the original EEG signal from a set of channels into a plurality of epochs, each of the plurality of epochs having an epoch duration length and an overlap increment, performing artifact reduction on the plurality of epochs to generate a plurality of artifact reduced epochs, and combining the plurality of artifact reduced epochs to generate a processed EEG recording, wherein each of the plurality of artifact reduced epochs overlaps an adjacent artifact reduced epoch to produce a continuous processed EEG recording without discontinuities in the processed EEG recording, wherein the plurality of artifact reduced epochs are combined using a weighted average wherein the weight of the epoch is proportional to a ratio of a distance to an epoch center;
overlaying the processed EEG report on the original EEG report to generate a combined EEG report, wherein an x-axis of the processed EEG report is aligned with an x-axis of the original EEG report, wherein the second plurality of channels of a y-axis of the processed EEG report is aligned with the first plurality of channels of a y-axis of the original EEG report, wherein the first plurality of channels of the original EEG report are equal to the second plurality of channels of the processed EEG report, wherein each of the x-axis of the processed EEG report and the original EEG report is time, and wherein the processed EEG report and the original EEG report match the time on the x-axis;
displaying the combined EEG report on a display screen of a monitor, the monitor in communication with the processor, wherein the processed EEG report is visually distinctive from the original EEG report, wherein an activity at a specific time on one channel of the first plurality of channels of the original EEG report is identifiable on a corresponding channel of the second plurality of channels of the processed EEG report at the specific time, wherein the amplitudes of the signals for the original EEG report are contained within each of the plurality of first channels in order to prevent overlapping of the signals and the amplitudes of the signals for the processed EEG report are contained within each of the plurality of second channels in order to prevent overlapping of the signals.

2. The method according to claim 1 wherein the artifact reduction is for at least one of muscle artifact, eye movement artifact, electrical artifact, heartbeat artifact, tongue movement artifact, and chewing artifact.

3. The method according to claim 1 wherein the combined EEG report comprises the processed EEG report having a first color and the original EEG report having a second color different than the first color.

4. The method according to claim 1 wherein the combined EEG report comprises the processed EEG report having a first style and the original EEG report having a second style different than the first style.

5. The method according to claim 1 further comprising switching from a display of the combined EEG report to a display of only the processed EEG report.

6. The method according to claim 1 wherein the overlay of the processed EEG report on the original EEG report has the overlay of each channel of the second plurality of channels of the processed EEG report within each corresponding channel of the first plurality of channels of the original EEG report.

7. (canceled)

8. A method for displaying EEG data, the method comprising:
generating an original EEG report from an EEG signal, the original EEG report generated from an EEG machine comprising a plurality of electrodes and processor, the original EEG report comprising a first plurality of channels;
performing artifact reduction on the original EEG signal to generate a processed continuous EEG report, the processed continuous EEG report comprising a second plurality of channels, wherein performing artifact reduction comprises partitioning the original EEG signal from a set of channels into a plurality of epochs, each of the plurality of epochs having an epoch duration length and an overlap increment, performing artifact reduction on the plurality of epochs to generate a plurality of artifact reduced epochs, and combining the plurality of artifact reduced epochs to generate a processed EEG recording, wherein each of the plurality of artifact reduced epochs overlaps an adjacent artifact reduced epoch to produce a continuous processed EEG recording without discontinuities in the processed EEG recording, wherein the plurality of artifact reduced epochs are combined using a weighted average wherein the weight of the epoch is proportional to a ratio of a distance to an epoch center;
overlaying the processed continuous EEG report on the original EEG report to generate a combined EEG report, wherein an x-axis of the processed continuous EEG report is aligned with an x-axis of the original EEG report, wherein the second plurality of channels of a y-axis of the processed continuous EEG report is aligned with the first plurality of channels of a y-axis of the original EEG report, wherein each of the x-axis of the processed continuous EEG report and the original EEG report is time, wherein the first plurality of channels of the original EEG report are equal to the second plurality of channels of the processed continuous EEG report, and wherein the processed EEG report and the original EEG report match the time on the x-axis;
displaying the combined EEG report on a display screen of a monitor, the monitor in communication with the processor, wherein the processed continuous EEG report is visually distinctive from the original EEG report, wherein an activity at a specific time on one channel of the first plurality of channels of the original EEG report is identifiable on a corresponding channel of the second plurality of channels of the processed continuous EEG report at the specific time, wherein the amplitudes of the signals for the original EEG report are contained within each of the plurality of first channels in order to prevent overlapping of the signals and the amplitudes of the signals for the processed EEG report are contained within each of the plurality of second channels in order to prevent overlapping of the signals.

9. The method according to claim 8 wherein the artifact reduction is for at least one of muscle artifact, eye movement artifact, electrical artifact, heartbeat artifact, tongue movement artifact, and chewing artifact.

10. The method according to claim 8 wherein the combined EEG report comprises the processed EEG report having a first color and the original EEG report having a second color different than the first color.

11. The method according to claim 8 wherein the combined EEG report comprises the processed EEG report having a first style and the original EEG report having a second style different than the first style.

12. (canceled)

13. The method according to claim 8 wherein the overlay of the processed EEG report on the original EEG report has the overlay of each channel of the second plurality of channels of the processed EEG report within each corresponding channel of the first plurality of channels of the original EEG report.

14. (canceled)

15. The method according to claim 8 further comprising switching from a display of the combined EEG report to a display of only the processed continuous EEG report.

16. The method according to claim 15 further comprising switching from the display of the processed continuous EEG report to a display of only the original EEG report.

17. A system for displaying EEG data, the system comprising:
a patient component comprising a plurality of electrodes for generating an EEG signal;
an EEG machine component comprising an amplifier and a processor, the processor configured to generate an original EEG report from an EEG signal, the original EEG report comprising a first plurality of channels, the processor configured to perform artifact reduction on the original EEG signal to generate a processed EEG report, the processed EEG report comprising a second plurality of channels, wherein the processor is configured to partition the original EEG signal from a set of channels into a plurality of epochs, each of the plurality of epochs having an epoch duration length and an overlap increment, the processor is configured to perform artifact reduction on the plurality of epochs to generate a plurality of artifact reduced epochs, and the process or is configured to combine the plurality of artifact reduced epochs to generate a processed EEG recording, wherein each of the plurality of artifact reduced epochs overlaps an adjacent artifact reduced epoch to produce a continuous processed EEG recording without discontinuities in the processed EEG recording, wherein the plurality of artifact reduced epochs are combined using a weighted average wherein the weight of the epoch is proportional to a ratio of a distance to an epoch center, and the processor configured to overlay the processed EEG report on the original EEG report to generate a combined EEG report, wherein an x-axis of the processed EEG report is aligned with an x-axis of the original EEG report, wherein the second plurality of channels of a y-axis of the processed EEG report is aligned with the first plurality of channels of y-axis of the original EEG report, wherein the first plurality of channels of the original EEG report are equal to the second plurality of channels of the processed EEG report, wherein each of the x-axis of the processed continuous EEG report and the original EEG report is time, and wherein the processed EEG report and the original EEG report match the time on the x-axis; and
a display screen of a monitor for displaying the combined EGG report wherein the processed EEG report is visually distinctive from the original EEG report, wherein an activity at a specific time on one channel of the first plurality of channels of the original EEG report is identifiable on a corresponding channel of the second plurality of channels of the processed EEG report at the specific time, the display screen in communication with the processor, wherein the amplitudes of the signals for the original EEG report are contained within each of the plurality of first channels in order to prevent overlapping of the signals and the amplitudes of the signals for the processed EEG report are contained within each of the plurality of second channels in order to prevent overlapping of the signals.

18. The system according to claim 17 wherein the artifact reduction is for at least one of muscle artifact, eye movement artifact, electrical artifact, heartbeat artifact, tongue movement artifact, and chewing artifact.

19. The system according to claim 17 wherein the combined EEG report comprises the processed EEG report having a first color and the original EEG report having a second color different than the first color.

20. The system according to claim 17 wherein the combined EEG report comprises the processed EEG report having a first style and the original EEG report having a second style different than the first style.
